(19) United States
(12) Patent Application Publication
(10) Pub. No.: US 2005/0053553 A1
(43) Pub. Date: Mar. 10, 2005

(54) Combined doses of formoterol and fluticasone

(76) Inventors: Thomas Nilsson, Mariefred (SE); Mattias Myrman, Stockholm (SE); Claes Friberg, Akers Styckebruk (SE); Sven Calander, Strangnas (SE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/703,505

(22) Filed: Nov. 10, 2003

Related U.S. Application Data
(60) Provisional application No. 60/500,262, filed on Sep. 5, 2003.

(30) Foreign Application Priority Data
Jun. 19, 2003 (SE) 0301816-5
Nov. 6, 2003 (SE) 0302939-4

(51) Int. Cl. A61L 9/04; A61K 9/14; A61K 31/58

(52) U.S. Cl. 424/46; 514/172

(57) ABSTRACT

The present invention discloses a method and pharmaceutical dry powder combined doses for administration by inhalation of metered dry powder combined doses of finely divided dry medication doses. Formoterol and fluticasone are selected medicaments for forming the combined doses. Metered dry powder medicinal combined doses comprising separately metered deposits of medicinally effective quantities of each of the selected medicaments are prepared, in which the sum of the metered deposits constitutes the metered quantities of powder of the combined doses and the medicinal combined doses are introduced into an adapted inhaler device for a generally simultaneous or sequential prolonged delivery of the medicinal combined doses during the course of a single inhalation by a user, such that each one of the administered medicinal combined doses is composed of a high proportion of de-aggregated fine particles of the selected medicament or medicaments and directed to a selected location in the lungs.

The present application is related to pending applications U.S. Ser. No. 10/603,818 filed Jun. 26, 2003 and provisional application U.S. Ser. No. 60/500 262 filed Sep. 9, 2003.

### TECHNICAL FIELD

The present invention relates to combined doses of medicaments for administration by an oral inhalation. In particular, combined doses of formoterol and fluticasone are packaged to fit a new method of aerosolizing selected combined doses into air and more particularly, the invention relates to combinations of separate dry powder entities of medicaments constituting the combined doses intended for administration by a single inhalation.

### BACKGROUND

Asthma and chronic obstructive pulmonary disease (COPD) affect more than 30 million people in the United States. More than 100,000 deaths each year are attributable to these conditions. Obstruction to airflow through the lungs is the characteristic feature in each of these airway diseases, and the medications utilized in treatment are often similar.

Up to 5% of the US population suffers from asthma, a respiratory condition characterized by airway inflammation, airway obstruction (at least partially reversible), and airway hyperresponsiveness to such stimuli as environmental allergens, viral respiratory-tract infections, irritants, drugs, food additives, exercise, and cold air. The major underlying pathology in asthma is airway inflammation. Inflammatory cell—eosinophils, CD4+ lymphocytes, macrophages, and mast cells—release a broad range of mediators, including interleukins, leukotrienes, histamine, granulocyte-colony-stimulating factor, and platelet aggregating factor. These mediators are responsible for the bronchial hyperreactivity, bronchoconstriction, mucus secretion, and sloughing of endothelial cells.

Chronic obstructive pulmonary disease (COPD) is a widespread chronic lung disorder encompassing chronic bronchitis and emphysema. The causes of COPD are not fully understood. Experience shows that the most important cause of chronic bronchitis and emphysema is cigarette smoking. Air pollution and occupational exposures may also play a role, especially when combined with cigarette smoking. Heredity also causes some emphysema cases, due to alpha1 anti-trypsin deficiency.

Chronic bronchitis is caused by excess mucus production in the lungs causing infection, which in turn causes inflammation and swelling, thus narrowing the bronchial tubes. This narrowing impedes airflow in and out of the lungs, causing shortness of breath. The condition usually begins with intermittent tracheobronchitis; however, repeated attacks occur until the disease and its symptoms persist continuously. If left untreated or if the patient continues to smoke, chronic bronchitis can lead to emphysema.

Administration of asthma drugs by an oral inhalation route is very much in focus today, because of advantages offered like rapid and predictable onset of action, cost effectiveness and high level of comfort for the user. Dry powder inhalers (DPI) are especially interesting as an administration tool, compared to other inhalers, because of the flexibility they offer in terms of nominal dose range, i.e. the amount of active substance that can be administered in a single inhalation. So far most development efforts have been directed towards producing effective drugs and formulations for specific abnormal conditions and not so much towards developing combined dose metering, forming methods and a suitable delivery device, i.e. the inhaler.

When inhaling a combined dose of dry medication powder it is important to obtain by mass a high fine particle fraction (FPF) of particles with an aerodynamic size preferably less than 5 μm in the inspiration air. The majority of larger particles does not follow the stream of air into the many bifurcations of the airways, but get stuck in the throat and upper airways. It is not uncommon for prior art inhalers to have an efficacy of 10-20% only, i.e. only 10-20% of the metered dose by mass is actually delivered as particles with an aerodynamic size less than 5 μm. Since most drugs may have undesirable side effects, e.g. steroids delivered to the system, it is important to keep the dosage to the user as exact as possible and to design the delivery system, e.g. an inhaler, such that the efficacy becomes much higher than 10-20%, thereby reducing the required amount of drug in the dose. Common, serious adverse effects of corticosteroids are osteoporosis, growth retardation, candidiasis and muscle injuries. Common, serious adverse effects of beta2-agonists are tremor, palpitations, headache, dizziness and oropharyngeal irritation.

In an article in Journal of Aerosol Medicine, Volume 12, Supplement 1, 1999, Pp. S-33-S-39 the authors Pavia and Moonen report clinical studies comparing therapy efficacy of a “soft mist inhaler” Respimat from Boehringer Ingelheim KG with that of an MDI. The studies show that the Respimat gives at least the same therapeutic bronchodilating effect as the MDI but using only half or less of the dosage in the MDI. The Respimat produces a slow-moving cloud of medicament droplets with a high fine particle fraction in a prolonged dose delivery taking in the order of one second, which reduces the deposition in the oropharynx and raises the topical delivery to the correct site of action in the lung. The challenge of developing inhalers capable of producing a delivered dose with a high fine particle fraction in a prolonged dose delivery is discussed in another article in Journal of Aerosol Medicine, Volume 12, Supplement 1, 1999, Pp. S-3-S-8 by the author Ganderton.

Interestingly, research during the past decade into respiratory diseases, their prophylaxis and treatment, has shown conclusively that simultaneous administration of combinations of different medicaments may improve the clinical condition of patients considerably. See for instance NIH, National Heart, Lung, and Blood Institute “Guidelines for the Diagnosis and Management of Asthma” NIH Publication No. 97-4051 July 1997, where a combined use of a long-acting beta2-agonist and a corticosteroid drug is recommended, formoterol and fluticasone being mentioned as good examples of substances of the two groups. The document stresses that it is important to reduce the adverse effects of the medicaments in general and inhaled corticosteroids in particular by reducing to a minimum the dosage, which still keeps the inflammation under control. At the time when these guidelines were compiled no medical products were available offering comprehensive combined medication together with suitable administration tools, at least not to the American public. The only possibility at the time was to combine by prescribing two different medicaments, preferably for inhalation, one from each group and separate inhalers for administration. This method of treatment was well known to practitioners at the time. Several studies in the mid-1990's have shown that by adopting a combined treatment it has been possible to reduce the dose of steroid compared to using the steroid as background treatment and a short-acting beta2-agonist as rescue medicine, besides improving lung function and reducing severity and frequency of attacks of dyspnoea.

For instance, in Switzerland patients diagnosed with asthma have been prescribed FORADIL (formoterol, a bronchodilating substance) together with PULMICORT (budesonide, an anti-inflammatory steroid) since the 1980's for treatment of their asthma. Until recently, however, different asthma medicaments have generally been administered separately, in sequence or by separate routes, not in compositions comprising more than one active ingredient. However, there are several published patent applications and approved patents teaching methods of treating respiratory disorders like asthma and chronic obstructive pulmonary disease (COPD) as well as pharmacologic compositions of different biological and chemical substances for this purpose, where the combinations offer overall advantages in the treatment of these conditions. See for instance EP 0416950B1 “Medicaments”, EP 0416951B1 “Medicaments comprising salmeterol and fluticasone”, EP 0613371B1 “New combination of formoterol and budesonid”, WO 98/15280 “New combination”, WO 00/48587 “Combinations of formoterol and fluticasone propionate for asthma”, WO 01/70198A1 “Stabilized dry powder formulations”, WO 01/78737A1 “Medical combinations comprising formoterol and budesonid”, WO 01/78745A1 “Medical combinations comprising formoterol and fluticasone propionate”, WO 02/28368A1 “New combination for the treatment of asthma”, WO 03/013547A1 “Pharmaceutical composition comprising salmeterol and budesonid for the treatment of respiratory disorders”. However, the quoted documents deal with aspects of formulating, processing, stabilizing and using mixtures of at least two ingredients. The mixing ratios between active ingredients and compositions thereof including suitable carriers, solvents and excipients are generally focused upon, not methods of administration or devices for that purpose.

A further document WO 01/78735, Sanders et al., claims a method of treating a respiratory disorder by administering an effective amount of the active ingredients formoterol and fluticasone separately, sequentially or simultaneously, provided that the ingredients comprise separate compositions. A dry powder inhaler containing formoterol and fluticasone in separate compositions is also claimed. However, Sanders et al. fail to teach how the skilled person should perform the method. Sanders goes on to teach that each of the active ingredients should be administered as separate compositions preferably once or twice daily. The document discloses that the claimed method may comprise an improvement of from 35-50% (in glucocorticoid receptor translocation into the nucleus) over known combination therapies, but no relevant information is given as to why the claimed method is superior and novel in relation to prior art, e.g. as exemplified in the previously mentioned documents. Further, no distinctive characteristics of the dry powder inhaler are disclosed, separating the inhaler from prior art inhalers.

A common denominator for the quoted documents is that they have as their first objective to simplify and improve asthma therapy for the user. A simpler, twice daily administration by inhalation of well-known, well-documented medicaments, one of which selected to address symptoms of broncho-constriction and the other to address an underlying inflammation of the bronchi, has proved in clinical testing to result in high user acceptance and compliance with a prescribed dosing regimen. The results of this therapy are in many reports compared with therapy using only the one or the other medicament, sometimes with increased dosages, or compared to separate prescriptions of said medicaments, but without specific instructions to the user on how to combine the administration of the two medicaments to achieve the best effect.

It comes as no surprise to a person of ordinary skill in the art that combining two well-documented medicaments, one to give quick relief of symptoms and the other to treat the cause in the long term, would be a good idea. The quoted documents all teach compositions of a beta2-agonist, preferably a long-acting bronchodilating drug with fast onset like formoterol, and a corticosteroid, i.e. an anti-inflammatory drug e.g. budesonide or fluitcasone propionate, in mixtures using effective amounts of the drugs and varying ratios between drugs depending on the condition, age, sex etc of the patient. The disclosed inventions in the quoted documents rely on existing MDI or DPI inhalers to do the job of delivering the medicament mixtures using a single inhaler. The documents also teach various techniques of combining two drugs in order to simplify self-therapy for asthmatics. The disclosed techniques range from mixing the drugs in various ways into an indivisable medicament to supplying medical kits composed of separately packaged doses for insertion in separate inhalers for separate, sequential delivery of the selected drugs. In the latter case it is difficult to see where the improvement for the user is lying.

None of the quoted documents indicate that the claimed medicament composition offers a therapeutic benefit, or quote clinical studies in support of such benefits, in comparison with separate, sequential delivery of the equivalent active medicaments. On the contrary, several documents teach that there is no therapeutic difference between delivering the active medicaments substantially simultaneously, sequentially or separately.

Furthermore, none of the quoted documents discusses in depth the importance of formulating a dry powder medicament for inhalation, e.g. the claimed compositions, such that an optimum distribution of particle aerodynamic diameters for optimum therapeutic effects from the selected drugs are arrived at. Also, there is no recommendation as to an order in which the different medicament doses, if physically separated, should be delivered to an inhaling user, presumably because a concept of delivering, in a single inhalation, combined doses composed of separate, individual doses of each medicament is unknown in prior art. Likewise, a concept of cutting back the quantities of active ingredients in the combined doses by implementing a giant increase in efficacy in the delivered dosage by adopting a prolonged dose delivery is also unknown in prior art.

The preferred embodiment of the inventions of the quoted documents is a mixture of the active drugs involving preferred prior art methods of preparing combined doses by mixing the ingredients. It is, however, difficult to mix dry medicament powders and optional excipients in a certain proportion consistently. The proportions in such a metered combined dose cannot easily be controlled, because the ratio of medicaments in an individual, combined dose depends significantly on the particle forces existing in each medicament powder, between particles of different medicaments and between medicament powders and dose packaging materials. Hence, actual variations in the ratio between active ingredients from combined dose to combined dose may be too large, causing serious problems if a potent ingredient is delivered in a higher or lower amount than expected.

Formoterol, a beta2-agonist, is a bronchodilator, which has been used with great success for more than 20 years in the treatment of asthma. It has proved to be a long-acting, potent drug with a fast onset and is widely used in the form of its fumarate salt. Different enantiomers of formoterol exist, e.g. RR, SS, SR, and RS with rather different efficacies as bronchodilators. Thus the recommended dosage of formoterol must be adjusted depending on which enantiomers are present and in what ratios in any particular formulation of formoterol. Formoterol is preferred by many asthmatics because a puff of the drug provides immediate relief during an attack of asthma. Formoterol as with all beta2-agonists, has no significant effect on underlying inflammation of the bronchi. Fluticasone on the other hand, especially fluticasone propionate, is an anti-inflammatory corticosteroid, which during the past two decades has proven to be a very successful and potent drug in reducing inflammation of nasal passages and bronchial tissue to make breathing easier. However, fluticasone propionate, like other anti-inflammatory steroids, does not have an immediate relief for a person suffering an asthma attack, but the drug will help to manage the inflammation and reduce the severity and number of exacerbations, if taken regularly.

National health-care institutions in most countries have been slow to actively promote the use of combined therapy, in the early days because of unfounded fear, as it turned out, of negative long-term side effects from the beta2-agonist, although in the last decade combined treatment has been listed as an open option for physicians in treating asthma patients. Thus, the full potential has not been realized of the obvious advantages, which may be achieved in a physician-controlled therapy using a combination of a bronchodilator and an anti-inflammatory drug in management of asthma and COPD. A reason for the slowness has been a lack of understanding among researchers and scientists of the complex mechanisms of airways diseases. Today, although much remains to be learned about asthma and COPD, many clinical tests have shown conclusively that combination therapy is working and provides good therapeutic results for many asthmatics.

Thus, there is a need for improvements regarding methods of treating respiratory disorders using combined, consistently metered doses of formoterol and fluticasone for co-ordinated administration by inhalation, using a new type of inhaler device.

### SUMMARY

The present invention discloses a method for the administration by inhalation of co-ordinated, metered, combined doses of finely divided dry powders of formoterol and fluticasone by means of an adapted inhaler designed for prolonged delivery of the combined doses. Metered dry powder medicinal combined doses are prepared comprising separately metered deposits of formoterol, including pharmaceutically acceptable salts, enantiomers, racemates, hydrates, solvates or mixtures thereof, and fluticasone, including pharmaceutically acceptable salts, enantiomers, racemates, hydrates, solvates or mixtures thereof, in suitable quantities and ratios, optionally including diluents or other excipients. “Formoterol” refers hereinafter to all the various chemical forms of the active substance, which are suitable for an intended therapeutic effect and particularly to formoterol fumarate. “Fluticasone” refers hereinafter to all the various chemical forms of the active substance, which are suitable for an intended therapeutic effect and in particular to fluticasone propionate. Because of the potency of the respective drugs it may be necessary to dilute the active substances, formoterol, A and fluticasone, B, separately using a pharmacologically acceptable diluent or excipient in order to secure the correct amounts as well as the ratio between the active substances, A and B, in the formed combined doses. The very small, individual quantities of active substances, A and B respectively, may be tightly controlled by careful metering of each entity of deposited powder, A′ and B′ respectively, constituting the combined doses. Hence, the sum of the metered entities constitutes the metered quantities of powder of the combined doses.

A user introduces the medicinal combined doses comprising the separated powder entities of formoterol and fluticasone into an adapted inhaler device for a prolonged delivery of the combined doses during the course of a single inhalation. Delivery of the separated entities of powder deposits of formoterol and fluticasone is preferably arranged to be sequential and more preferably such that formoterol is delivered first and fluticasone shortly after, so that formoterol may reach deeper into the lung for topical action and fast onset, while fluticasone may be topically deposited in the lung for best effect with as little systemic effect as possible. The delivered doses are composed of a high proportion of de-aggregated fine particles of the selected medicaments respectively, although the particle flows are preferably separated in time, whereby an intended prophylactic, therapeutic and psychologic effect on the user is achieved.

Furthermore, pharmaceutical dry powder combined doses of formoterol and fluticasone are disclosed. The doses are adapted for inhalation, for the prophylaxis or treatment of a respiratory disorder in a user. The pharmaceutical dry powder combined doses are prepared comprising separate entities of metered deposits of medicinally effective quantities of formoterol and fluticasone respectively, optionally including diluents or excipients, where the sum of the entities constitutes the metered quantities of powder in the pharmaceutical, combined doses suitable for being introduced into an adapted inhaler device.

The present method is set forth by the independent claim 1, 12 and the dependent claims 2 to 6 and 13, and pharmaceutical combined doses are set forth by the independent claim 7 and the dependent claims 8 to 11.

### BRIEF DESCRIPTION OF THE DRAWINGS

The invention, together with further objects and advantages thereof, may best be understood by referring to the following detailed description taken together with the accompanying drawings, in which:

FIG. 1 illustrates in top and side views a first embodiment of combined doses comprising two medicament entities deposited in separate compartments onto a dose bed;

FIG. 2 illustrates in top and side views a second embodiment of combined doses comprising three medicament entities deposited in separate compartments onto a dose bed;

FIG. 3 illustrates in top and side views a third embodiment of combined doses comprising two parallel medicament entities deposited onto a dose bed;

FIG. 4 illustrates in top and side views a fourth embodiment of combined doses comprising several medicament entities and separating excipient entities deposited onto a dose bed;

FIG. 5 illustrates in top and side views a fifth embodiment of combined doses comprising four medicament entities and separating excipient entities deposited onto a dose bed;

FIG. 6 illustrates in top and side views a sixth embodiment of combined doses comprising two parallel medicament entities deposited on top of one another onto a dose bed;

FIG. 7 illustrates in top and side views a seventh embodiment of combined doses comprising two medicament entities deposited on top of one another onto a dose bed, but separated by a deposited excipient entity;

FIG. 8 illustrates in top and side views another embodiment of combined doses comprising two medicament entities separately deposited onto a dose bed;

FIG. 9 illustrates in top and side views yet another embodiment of combined doses comprising two medicament entities separately deposited onto a dose bed, but with some degree of overlap;

FIG. 10a illustrates in a sectional view an example of combined doses comprising two medicament entities deposited on top of one another but separated by a deposited excipient entity onto a dose bed and adjacent to the combined doses a nozzle in a starting position before the combined doses are released;

FIG. 10b illustrates in a sectional view an example of combined doses comprising two medicament entities deposited on top of one another but separated by a deposited excipient entity onto a dose bed and adjacent to the combined doses a nozzle in a relative motion sucking up the powder particles to be dispersed into the air stream;

### DETAILED DESCRIPTION

The present invention discloses a new combination of active asthma drugs comprising two coordinated, metered, combined doses of the medicaments formoterol, particularly formoterol fumarate, and fluticasone, in particular fluticasone propionate. In a further aspect, the invention discloses a new therapeutic method of treating respiratory diseases like asthma by delivering such coordinated combined doses by an inhalation route to a user of a dry powder inhaler (DPI). “Asthma” is used in this document as a generic term for the different respiratory disorders known in the field of medicine.

In the context of this application the word “medicament” is defined as a pharmacologic substance, which comprises at least one chemically or biologically active agent. Further, a medicament may exist in a pure form of one or more pure active agents, or a medicament may be a compound comprising one or more active agents, optionally formulated together with other substances, e.g. enhancers, carriers, diluents or exipients. Hereinafter, the term “excipient” is used to describe any chemical or biologic substance mixed in with a pure active agent for whatever purpose. In this document, only medicaments in dry powder form are discussed. Formoterol and fluticasone respectively are in this document generic terms for the respective active chemical substances including pharmaceutically acceptable salts, enantiomers, racemates, hydrates, solvates or mixtures thereof, which have a desired, specific, pharmacologic and therapeutic effect.

A “dose bed” is henceforth defined as a member capable of harboring metered combined doses comprising one or more entities of dry powders, where the combined doses are intended for delivery to a user of a DPI in a single inhalation performed by the user. Different types of pharmaceutical blister packs or capsules are included in the term “dose bed”. In the present invention combined doses for treating asthma comprise metered, deposited entities of formoterol and fluticasone respectively, optionally including excipients. The dose bed may be divided in two areas or incorporate two compartments, i.e. cavities of suitable volume, intended for deposited entities of dry powders of formoterol and fluticasone respectively. In a preferred embodiment the combined doses are packaged for a, prolonged delivery, i.e. the delivery period for the combined doses is in a range from 0,01 to 6 s, usually in a range from 0,1 to 2 seconds, delivery taking place sometime during the course of an inhalation as controlled by a purposefully designed DPI, adapted for combined doses. Advantageously, such a DPI adopts an Air-razor method of gradual aerosolization of the combined doses by introducing a relative motion between an air-sucking nozzle and the powder doses. Advantages of a prolonged delivery of a dose for inhalation are disclosed in our U.S. Pat. No. 6,571,793 B1 (WO 02/24264 A1), which is hereby incorporated in this document in its entirety as a reference.

A preferred embodiment of metered combined doses use a dose bed split up in two separate compartments, where each compartment is intended for a metered deposition of a particular asthma medicament, in this case formoterol and fluticasone respectively and more particularly formoterol fumarate and fluticasone propionate. Each compartment containing a metered entity of a medicament powder may then be sealed, e.g. by foiling, such that the different medicaments in the different compartments of the dose bed cannot interact in any way and cannot be contaminated by foreign substances or moisture. Alternatively, a common foil may enclose both compartments, and sealing between compartments may be excluded if individual sealing is not a GMP or medicinal requirement. A dose carrier is normally engaged to carry at least one dose bed loaded with combined doses, whereby the dose carrier may be inserted into a DPI for administering the combined doses, e.g. sequentially, to a user in need of treatment. A suitable carrier of combined doses is disclosed in our U.S. Pat. No. 6,622,723 B1 (WO 01/34233 A1), which is hereby incorporated in this document in its entirety as a reference. However, a dose bed may be designed to act as a carrier, intended for direct insertion into a DPI. A suitable DPI for a continuous, prolonged dose delivery is disclosed in our U.S. Pat. No. 6,422,236 B1, which is hereby incorporated in this document in its entirety as a reference.

If complete physical separation of the deposited entities of the two medicaments making up the combined doses, is not required but some degree of overlap or mixing is acceptable from a physical, chemical and medical point of view, then other methods of separating the deposited entities may be implemented. Depending on what degree of mixing is permitted or in some cases desired, different ways of separating medicament entities must be adopted. For example, the dose bed may use separate indentations where different powders should be deposited, but flat target areas for separate deposits in a single plane on the dose bed are equally possible. In another embodiment the two medicaments are deposited sequentially dot-wise or string-wise onto two target areas of the dose bed. If necessary, to stop chemical or biological interaction or decomposition caused by, for example, adjacent medicament powders being incompatible, an isolating, biologically acceptable, inert substance like carbohydrates, e.g. glucose or lactose, may be deposited between the medicament entities. When the combined dose entities have been completely formed they are usually sealed from ingress of dirt and moisture by a foil covering the entire dose bed. A method of depositing microgram and milligram quantities of dry powders using electric field technology is disclosed in our U.S. Pat. No. 6,592,930 B2, which is hereby incorporated in this document in its entirety as a reference.

Forming combined doses comprising two medicaments in separate dry powder formulations may be done in different ways, known in prior art. The invention discloses that the finely divided powders to be included in the combined doses, i.e. formoterol and fluticasone respectively, need not be mixed or processed together prior to dose forming and, indeed, should be kept separated during dose forming as well as after the respective entities of the combined doses are formed and sealed. The medicament entities of the combined doses are thus kept separated on the dose bed by suitable methods, as described in the foregoing, until the combined doses are about to be delivered by an inhalation route to a user and thereby preferably delivered in sequence, separated in time and therefore not mixed in the inhaled air leaving the mouthpiece of the DPI.

The present invention offers inherent manufacturing advantages in comparison with prior art methods, which are based on mixing the active ingredients in bulk quantities, generally including diluents and/or carriers before forming doses. The consequence of this mixing step in the manufacturing process, apart from the regulatory problem of proving the mixture as such, is that many different blends of mixture must be made and verified to provide the correct ratios between the active ingredients in order to correspond to given therapeutic requirements, since different patients need different ratios, besides correct quantities. Disregarding the problem of verifying a mixture in bulk quantity, besides the problem of verifying the actual ratio between ingredients in each individual dose, a further consequence of the mixing step is the extra time required for producing, storing and verifying the mixture before and during the dose forming process. Also to be considered is the circumstance that it is not uncommon for active substances to have a limited period of stability, which is often even shorter when mixed with other active ingredients.

The present invention avoids all of these problems, since the active ingredients are kept separate, optionally in a mixture with excipient(s), all the way through the dose manufacturing process, and, in fact, during packaging, distribution and storing until the moment when the user has introduced the combined doses into an inhaler and starts to inhale. Furthermore, the ratio between the active ingredients represents no problem, since it is a result of the metered quantities of the respective active ingredients constituting the combined doses.

Although the medicament entities of the combined doses are separated on the dose bed until the doses are to be delivered by a DPI, it is perfectly possible according to alternative embodiments of the invention to suck up the doses more or less mixed into the inspiration air during inhalation. In one aspect the powder entities of the combined doses of formoterol and fluticasone may be sucked up simultaneously, partly or completely. The degree of mixing of the delivered powders leaving the DPI mouthpiece may vary between 0 and 100% depending partly on the design of the DPI and its suction system, partly on the physical relative positions between deposited powder entities on the dose bed and partly on the relation between the dose bed and the suction system. For instance, if fluticasone is deposited first onto a dose bed and formoterol is then deposited on top of the fluticasone, the powders will be mixed practically to 100% when sucked up.

In another aspect the powder entities of the combined doses may be sucked up sequentially, e.g. if the powder entities are accessed one at a time by the suction system of the DPI in the course of a single inhalation. Naturally, in that case, no mixing of powders will happen, since the delivery of the doses into inspiration air will be sequentially time separated.

In a third aspect, by selecting a pattern of physical positions and extensions in space of the deposited powder entities when forming the doses, it will be possible to tailor the delivery of the powders in the doses such that the medicament powders get mixed into inspiration air to a selected degree between 0 and 100%.

Methods of dose forming include conventional mass or volumetric metering and devices and machine equipment well known to the pharmaceutical industry for filling blister packs, for example. Also see European Patent No. EP 0 319 131 B1 and U.S. Pat. No. 5,187,921 for examples of prior art in volumetric and/or mass methods and devices for producing doses of medicaments in powder form. Electrostatic forming methods may also be used, for example as disclosed in U.S. Pat. Nos. 6,007,630 and 5,699,649. Any suitable method capable of producing metered microgram and milligram quantities of dry powder medicaments may be used. Even completely different methods may be applied to suit the different medicaments selected to be part of the combined doses to be produced. A dose may hold together in a more or less porous entity by action of van der Waals forces, electrostatic forces, electric forces, capillary forces etc interacting between particles and particle aggregates and the dose bed material.

Total mass in combined doses according to the present invention is typically in a range from 5 μg to 5 mg, but may extend to 50 mg. Regardless of which forming and filling method is being used for a particular medicament, it is important during dose forming to make sure that selected medicaments are individually metered and deposited onto their respective target areas or compartments of the dose bed. The target areas or compartments of the dose bed, which aggregate to hold combined doses, may be of a same size or different sizes. The shape of compartments is governed by physical constraints defined by the type of dose bed used. As an example, a preferred type of dose bed is an elongated strip of a biologically acceptable, inert material, e.g. plastic or metal, between 5 and 50 mm long and between 1 and 10 mm wide. The strip is further divided into separate target areas or compartments arranged along the length of the elongated strip. The dose bed or, if necessary each compartment, receives an individual seal, for instance in the form of a foil, in a step immediately subsequent to the dose forming.

An advantage of the present invention is that formoterol and fluticasone are selected on merits of their own for inclusion in combined doses, in disregard of whether or not the respective formulations are compatible with one another. Thus, the regulatory process before introducing combined doses of e.g. formoterol fumarate and fluticasone propionate on the market may be drastically simplified. Yet another advantage of the invention is the possibility of using pure, potent formoterol and fluticasone substances for inclusion in the combined doses, without any included excipients.

TABLE 1
Typical dosages of formoterol and fluticasone respectively in asthma
therapy

| Medicament active | Delivered dosage range | Delivered dosage range |
|---|---|---|
| agent | per dose (μg) | per day for adults (μg) |
| Formoterol | 1-50 | 1-100 |
| Fluticasone | 40-2000 | 50-5000 |

Combined doses are intended for administration in a single inhalation, either irregularly when need arises, or more typically as part of a daily management regime. The number of combined doses administered regularly may vary considerably depending on the type of disorder. Optimal dosages of formoterol and fluticasone respectively for prevention or treatment of respiratory disorders may be determined by those skilled in the art, and will vary with their respective potency and the advancement of the disease condition. Furthermore, factors associated with the individual undergoing treatment determine correct dosages, such as age, weight, sex etc. Depending on what are correct dosages per day and the number of planned administrations per day, the correct deposits by mass for the prepared medicaments may be calculated, such that metered deposits of each medicament entity to be included in the metered combined doses may be produced in a dose-forming step. In calculating a correct nominal deposit of mass for each medicament entity the fine particle fraction, i.e. particles having a mass median aerodynamic diameter (MMAD) less than 5 μm, per entity of the actual delivered doses must be taken into consideration. As discussed in the foregoing, the efficacy of inhalers differs considerably and it is thus important to include the expected efficacy of the chosen inhaler in the calculation of a suitable nominal mass in the deposited entity or entities. What constitutes suitable amounts of the two medicaments and the respective optimal masses of formoterol and fluticasone respectively depend on the factors described in the foregoing, but generally the inhaled formoterol mass, e.g. in the form of formoterol fumarate, per dose should be in a range from 1 to 50 μg, preferably between 2 to 40 μg and inhaled fluticasone, e.g. in the form of fluticasone propionate, per dose in a range from 40 to 2000 μg, preferably between 50 to 500 μg.

There is generally a medical need to direct the delivery, i.e. the deposition, of inhaled doses of a medicament to the optimum site of action, where the therapeutic effect is the best possible, in the airways or lungs, including the deep lung, either for a topical effect or for a systemic effect. Turning to the case in point, it is of course desirable to control the deposition of the combined doses of formoterol and fluticasone to their respective sites of action in the airways and lungs in order to get highest possible overall efficacy for each dose with a minimum of side effects. Aerodynamic particle size is a most important factor greatly influencing where in the airways and lungs particle deposition is likely to take place. From a target site point of view, it is therefore desirable to tailor the physical formulations of the respective medication powders in the combined doses in such a way that they result in an advantageous particle aerodynamic size distribution by mass in the delivered dose. The present invention makes it possible to deliver the combined doses, thus formulated, to the targeted sites of action. Available data indicate that for best performance, the AD (aerodynamic diameter) for fluticasone in the delivered dose should be in a range from 2 to 8 μm whereas AD for formoterol in the delivered dose should be in a range from 1 to 5 μm.

Another circumstance to consider is the order of delivery for the combined doses of the present invention. The first air to be sucked in by a person inhaling reaches deep into the peripheral lung and air sucked in thereafter fills up the lungs gradually. Generally, what this means is that powders intended for a peripheral lung deposition should be inhaled early in the inhalation cycle to maximize deposition in that area and powders intended for a central lung deposition should be inhaled somewhat later in the cycle to maximize deposition in the central lung. Since available data suggest that formoterol should preferably deposit in the peripheral lung area and fluticasone in the central lung area, a dose of formoterol should be the first to be sucked in followed by a dose of fluticasone. Under the proviso that an adapted DPI is at hand for a sequential delivery of the combined doses in the course of a single inhalation, the present invention refutes prior art and claims that sequential delivery of combined doses, i.e. a dose of formoterol first followed by a dose of fluticasone thereafter, is to be preferred compared to simultaneous delivery, e.g. combined doses in the form of a mixture. Compared to prior art the present invention presents a definite advantage regarding delivered dose efficacy and benefits for the user.

The present invention makes use of proven dry powder formulations of formoterol and fluticasone, particularly formoterol fumarate and fluticasone propionate, finely divided and adapted for separate deposition onto a common dose carrier, normally with no mixing of the two active substances. Combined doses thus formed may be introduced into an adapted dry powder inhaler (DPI) such that the medicament entities constituting the combined doses may be aerosolized and delivered in the inspiration air during the course of a single inhalation through a DPI by a user. Keeping the different medicament entities separated according to the invention may reduce the investment in time and resource necessary for getting the combined doses approved by the relevant regulatory bodies and released to the respective markets. For instance, no added substance to stabilize the combined doses will be needed and no testing to prove that an added substance is harmless needs to be performed.

The present invention differs from prior art inhalers and related combined dose delivery methods by providing combined doses comprising two co-ordinated, individually proven asthma medicaments in form of separately deposited entities onto a dose bed. The combined doses are therefore not a single composition of asthma medicaments constituting a single physical entity. The invention discloses combined doses comprising at least two co-ordinated physical medicament entities loaded onto a common dose carrier with an objective of providing more efficient treatment of asthma. Inserted into an adapted DPI, the combined doses will be aerosolized during a single inhalation by a user. Preferably, the entities of the combined doses of formoterol and fluticasone will be delivered sequentially or optionally more or less simultaneously into the inspiration air. Whether the combined doses of medicaments are aerosolized sequentially or simultaneously depends on the physical form of the combined doses, i.e. how the deposited medicament entities are interrelated, and on the type of inhaler used to administer the combined doses.

It is obvious that an inhaler, which instantaneously subjects all powders of the combined doses to a jet-stream of air will aerosolize the aggregated deposits more or less simultaneously, whereby the medicament powders, still more or less agglomerated, become mixed into the air leaving the mouthpiece. In contrast, an inhaler subjecting the combined doses to a jet stream gradually, like a moving tornado attacking adjacent corn fields, one after the other, thereby not attacking all of the powder entities of the combined doses instantly, may aerosolize the entities of the combined doses gradually and efficiently over time. An object of the invention is to offer better control of dose release and to facilitate a prolonging of dose delivery in order to produce a high fine particle fraction (FPF) in the delivered, combined doses. Another object of the invention is to achieve a high ratio of delivered, combined doses relative metered, combined doses. Although it is possible to successfully apply the invention to prior art inhalers, they tend to deliver the combined doses more or less mixed in too short a time, resulting in a poor FPF figure and low efficacy. On the other hand, a gradual, well-timed, sequential delivery of combined doses is possible using a new inhaler design where a relative movement is introduced between the combined doses and a suction nozzle through which the inspiration airflow is channeled. This arrangement utilizes the inhalation effort of the user to aerosolize the combined doses gradually for a prolonged period of dose delivery, thus using the power of the suction more efficiently and eliminating in most cases a need for external power to aerosolize the combined doses.

A powder Air-razor method is advantageously used for aerosolizing the medicament powder entities of the combined doses, the Air-razor providing de-aggregation and dispersal into air of the finely divided medication powders. By utilizing an effort of sucking air through a mouthpiece of an inhaler, said mouthpiece connected to a nozzle, the particles of the deposited medicament powders, made available to the nozzle inlet, are gradually de-aggregated and dispersed into a stream of air entering the nozzle. The gradual de-aggregation and dispersal is produced by the high shearing forces of the streaming air in connection with a relative motion introduced between the nozzle and the powder entities of the combined doses. In a preferred embodiment, the medicament powders are deposited onto a dose bed, such that the powder deposits occupy an area of similar or larger size than the area of the nozzle inlet. The nozzle is preferably positioned outside the area of deposits, not accessing the powder by the relative motion until the air stream into the nozzle, created by an applied suction, has passed a threshold flow velocity. Coincidental with the application of the suction or shortly afterwards the relative motion will begin such that the nozzle traverses the powder entities constituting the combined doses gradually. The high velocity air going into the nozzle inlet provides plenty of shearing stress and inertia energy as the flowing air hits the leading point of the border of the contour of the medicament entities, one after the other. This powder Air-razor method, created by the shearing stress and inertia of the air stream, is so powerful that the particles in the particle aggregates in the powder adjacent to the inlet of the moving nozzle are released, de-aggregated to a very high degree as well as dispersed and subsequently entrained in the created air stream going through the nozzle. If the medicament deposits have been made in separate compartments of the dose bed and individually sealed, then obviously the compartments must be opened up first so that the nozzle can access the deposited powder entities in each compartment when suction is applied. Naturally, this is also true if the deposits share a common seal without an individual seal for each deposited entity. An arrangement for cutting foil is disclosed in our Swedish patent publication SE 517 227 C2 (WO 02/24266 A1), which is hereby incorporated in this document in its entirety as a reference. Depending on how the entities are laid out on the dose bed, the nozzle will either suck up the powder entities sequentially or in parallel or in some serial/parallel combination.

The present invention improves the efficacy of formoterol/fluticasone dose delivery, compared to the best selling inhalers on the market today, by at least a factor of two and typically 2.5. This is accomplished by raising the FPF <5 μm in the delivered dose to more than 40%, preferably to more than 50%, by mass, compared to typically less than 30% for prior art inhalers. The implications of this vast improvement are much less adverse reactions in users, even to the point of eliminating the risk of death, due to high dosages of beta2-agonists or corticosteroids systemically or in the wrong parts of the airways.

Thus, the quality of asthma medicament delivery is dramatically improved compared to prior art performance, leading to important advances in delivering a majority of fine particles of the asthma medicaments of the combined doses to the intended target area or areas in the user's airways and lungs with very little loss of particles settling in the throat and upper airways. Administering asthma medicament combinations according to the present invention has a very positive therapeutic effect from a medical, psychological and social point of view on a host in need of asthma treatment with a coordinated combination of formoterol and fluticasone.

### DETAILED DESCRIPTIONS OF DRAWINGS

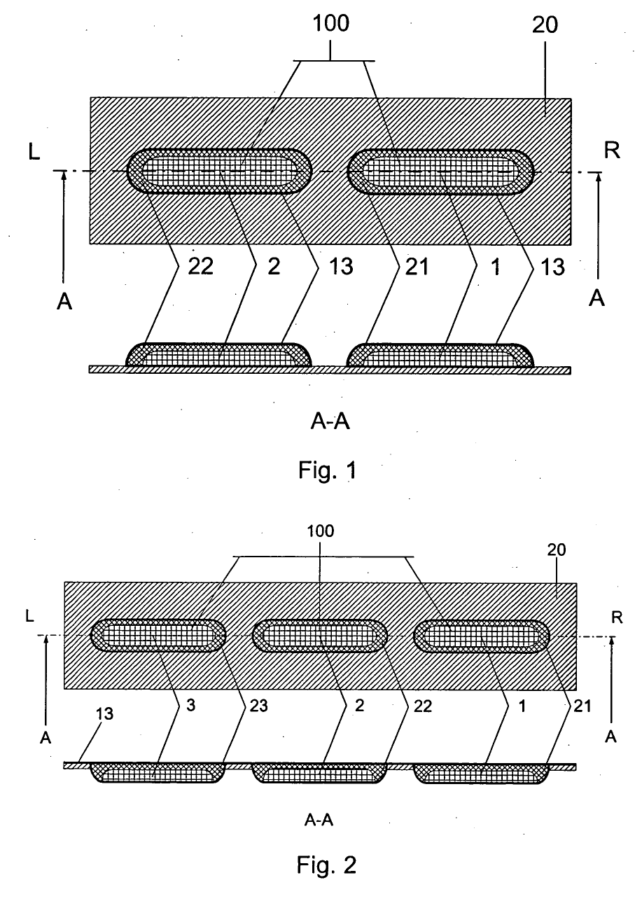

Referring to reference numbers 1-100 of the drawings wherein like numbers indicate like elements throughout the several views of ten different embodiments of combined doses comprising at least two deposited entities of two medicaments onto a dose bed as illustrated in FIGS. 1-10 presented here as non-limiting examples.

FIG. 1 illustrates combined doses 100 comprising two different medicament entities deposited, 1 and 2, in separate compartments 21 and 22 onto a dose bed 20, said compartments may be capsules or blisters or moldings in the dose bed. An individual seal 13 for each compartment guarantees that the medicament doses cannot be contaminated by foreign matter or by one another. The illustrated doses are intended for a sequential delivery taking place during a single inhalation.

FIG. 2 illustrates combined doses 100 comprising three different medicament entities, 1, 2 and 3 in separate compartments 21, 22 and 23 similar to FIG. 1, but arranged underneath the dose bed 20. Besides a different arrangement of compartments on the dose bed 20 and the respective seals 13, the main difference between FIG. 1 and FIG. 2 is that entity 3 consists of the medicament of entity 2. It is thus possible not only to administer two medicaments, but also to compose combined doses of two medicaments with a very high ratio of mass between them. The illustrated deposited entities are intended for a sequential delivery taking place during a single inhalation.

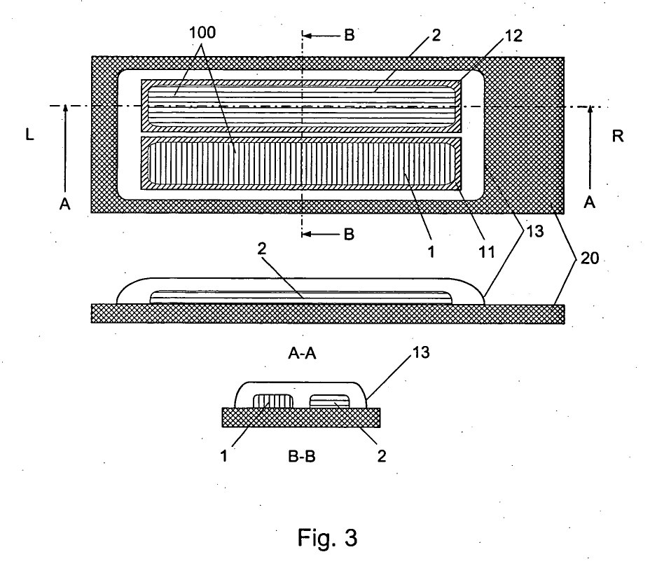

FIG. 3 illustrates combined doses 100 comprising two different medicament entities, 1 and 2, laid out in parallel strips onto separate target areas 11 and 12 respectively onto the dose bed 20. A common protective foil 13 protects the medicaments of the combined doses from being contaminated by foreign matters. The illustrated entities are intended for a fully simultaneous delivery of the two medicaments taking place during a single inhalation.

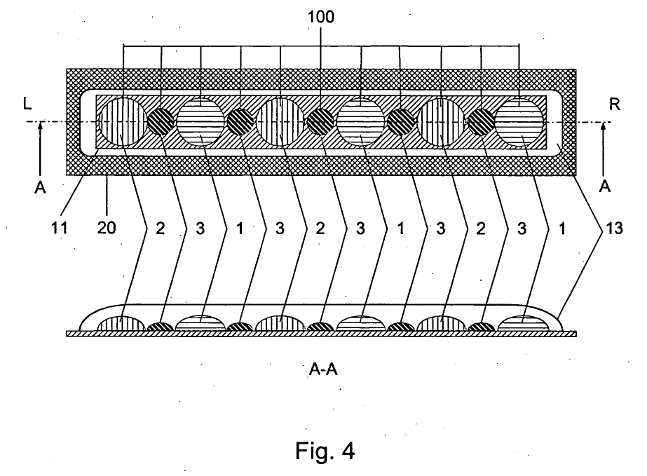

FIG. 4 illustrates combined doses 100 comprising two different medicaments, 1 and 2, each comprising several deposited entities separated by deposited entities of an inert excipient 3. The deposited entities are laid out in a string of spots onto a target area 11 on a dose bed 20. The entities share a common seal 13. The combined doses are intended for a sequential delivery of incorporated medicament and excipient entities, said delivery taking place during an inhalation. The excipient deposits help to minimize unintentional mixing of the medicaments. If some mixing of medicaments can be accepted, then the excipient entities may be left out altogether. Combined doses composed of spot entities may of course comprise more medicaments than two. The mass ratio between medicament doses may be easily set by controlling the ratio between the number of spot entities per medicament in combination with the size of the respective spot entities in terms of deposited mass. Naturally the spot entities need not necessarily be circular in shape, they may take an elongated or elliptical form, depending on which types of combined dose forming methods are used.

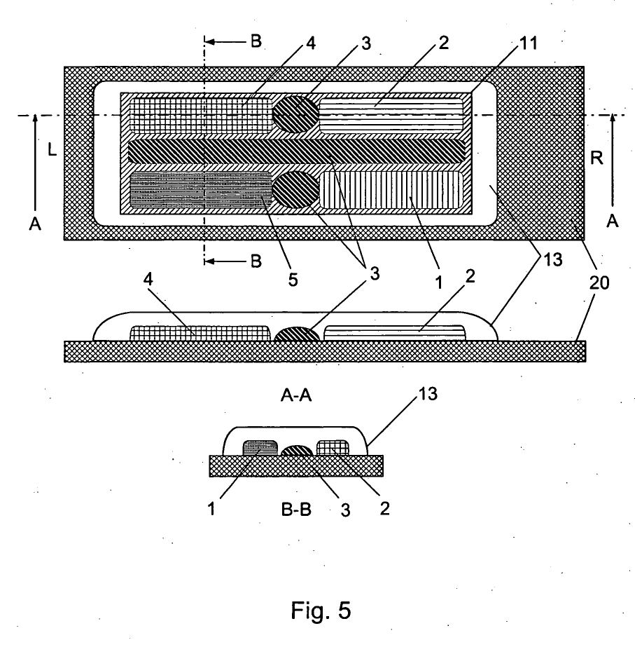

FIG. 5 illustrates combined doses 100 comprising deposited entities representing up to four different medicaments, 1, 2, 4 and 5, separated by deposited entities of an inert excipient 3. The deposited entities are laid out in two parallel groups of two entities per group lined up in strips onto a common target area 11 on a dose bed 20. The deposited entities share a common seal 13. The excipient deposited entities help to minimize unintentional interaction of the medicament doses. The combined doses are intended for a combined parallel/simultaneous and sequential delivery of incorporated medicament doses, said delivery taking place during a single inhalation.

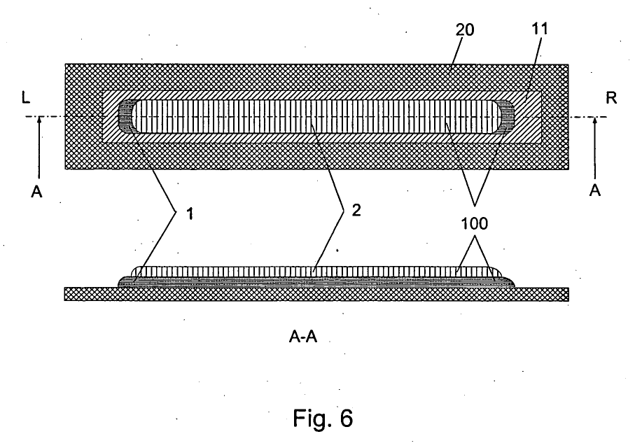

FIG. 6 illustrates combined doses 100 comprising two different medicament entities, 1 and 2, each comprising a strip of deposited powder, medicament 1 deposited onto a target area 11 of a dose bed 20 and medicament 2 deposited on top of the entity of medicament 1. This method of forming combined doses is an alternative to the ones previously disclosed and may be used when a certain level of interaction or mixing of the medicaments may be tolerated.

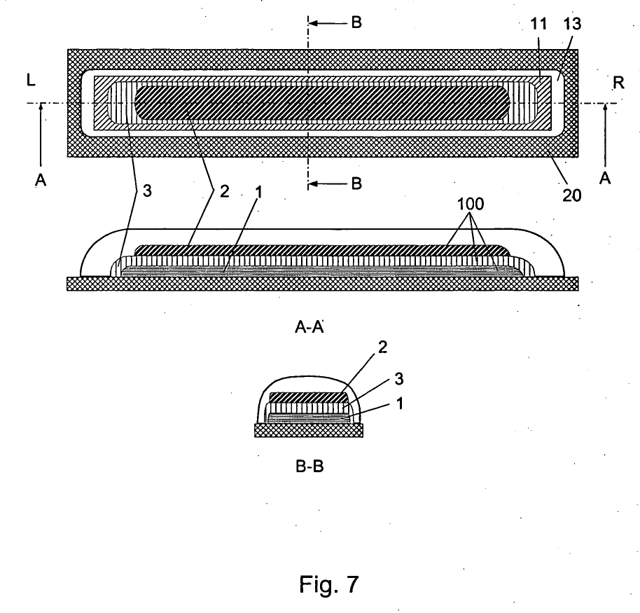

FIG. 7 illustrates combined doses 100 comprising two different medicament entities, 1 and 2, and an excipient entity 3, each comprising a strip of deposited powder. Medicament 1 is deposited onto a target area 11 of a dose bed 20 and excipient 3 is deposited onto medicament 1 to insulate medicament 1 from a deposit of medicament 2 on top of the deposited entity of excipient 3.

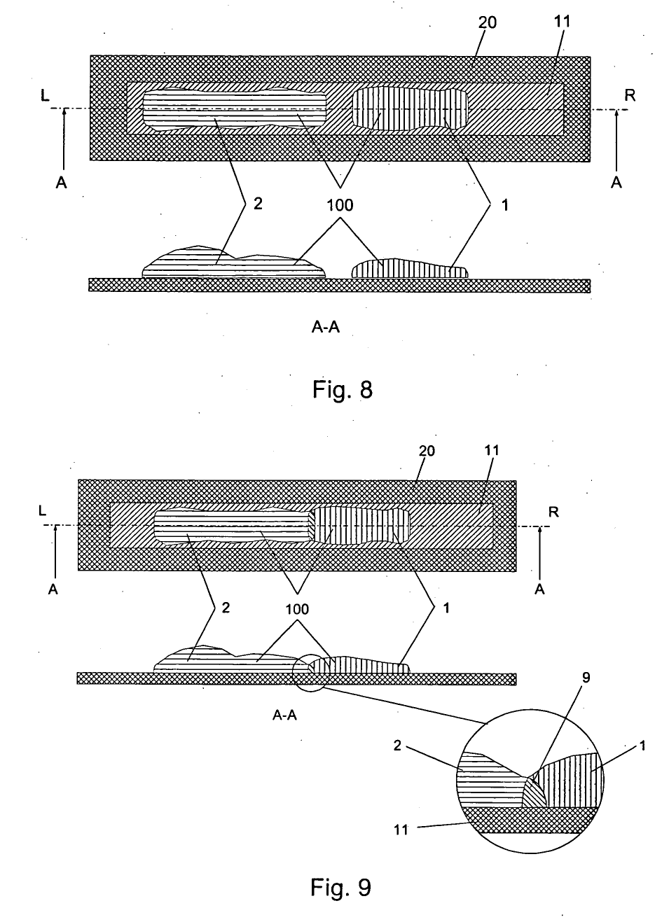

FIG. 8 illustrates combined doses 100 comprising two different medicament entities, 1 and 2, of somewhat irregular shapes but separately laid out onto a common target area 11 of the dose bed 20. The illustrated entities are intended for a sequential delivery of the two medicament doses taking place during an inhalation.

FIG. 9 illustrates combined doses 100 comprising two different medicament entities, 1 and 2, of somewhat irregular shapes but generally separately laid out onto a common target area 11 of the dose bed 20. The illustrated deposited entities overlap slightly, resulting in an arbitrary mixture 9. The deposits are intended for a mostly sequential delivery of the two medicament doses taking place during a single inhalation.

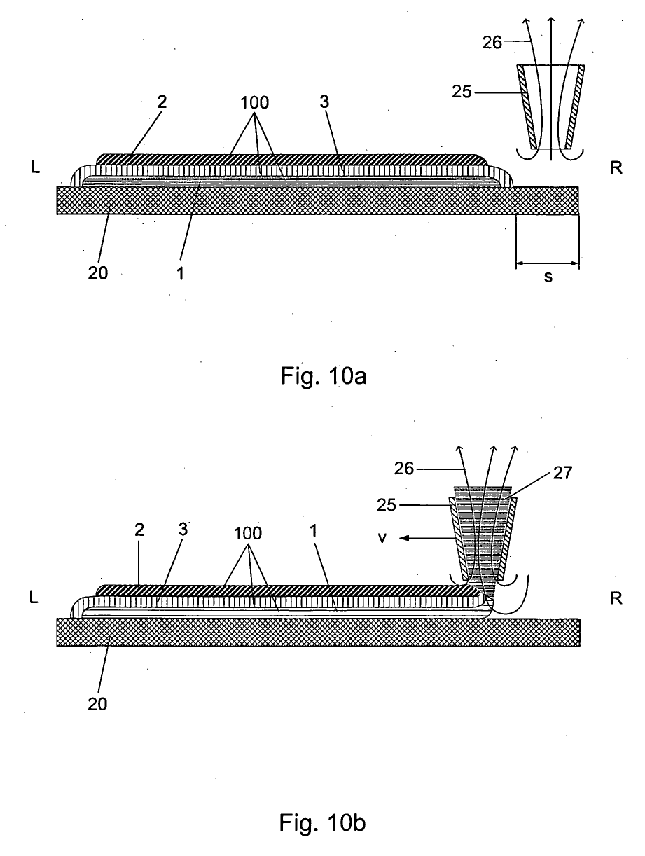

FIGS. 10a and 10b illustrate a delivery of combined doses 100 comprising two different medicament entities, 1 and 2, and an excipient entity 3, each comprising a strip of powder sequentially deposited in three different layers. A nozzle 25 with an established flow of air 26 going into the inlet is put in a relative motion, parallel to the dose bed 20, such that the nozzle passes over the combined doses beginning at the right side R and ending at the left side L of the dose bed. This Air-razor method results in a simultaneous, gradual delivery of medicament entities 1 and 2 together with the excipient entity 3. The powders of the entities are mixed into an aerosol 27 by the air flowing into the nozzle leading to simultaneous delivery of the two medicament doses and the excipient. This Air-razor method may be applied to all embodiments of the present invention and results in a simultaneous or sequential or a combined simultaneous/sequential delivery of all included medicament doses and optional excipients.

1. A method for the administration by inhalation of metered dry powder combined doses of finely divided dry medication powders by a dry powder inhaler device, comprising the steps of
selecting medicaments (a) and (b) for a forming of pharmaceutical, combined doses, where (a) stands for formoterol or a pharmaceutically acceptable salt, enantiomer, racemate, hydrate, or solvate including mixtures thereof, and (b) stands for fluticasone or a pharmaceutically acceptable salt, enantiomer, racemate, hydrate, or solvate including mixtures thereof, and where (a) and (b) may optionally further include excipients;
preparing metered dry powder medicinal combined doses comprising separately deposited entities of medicinally effective quantities of each of the medicaments onto selected target areas of a common dose bed, the sum of the deposited entities constituting the metered quantities of powder of the medicinal combined doses;
coordinating during preparation the entities of the combined doses such that, after introduction into an inhaler device adapted for a prolonged delivery, when suction is applied through the inhaler, the powders of each of the entities are gradually aerosolized, generally presenting a fine particle fraction, fpf at least 40%, preferably at least 50%, of delivered powder mass, whereby the entities of the combined doses are delivered to and deposited in the lung either simultaneously or separately in sequence, or in a combination thereof, during a single inhalation effort by a user.

2. The method according to claim 1, comprising the further step of
selecting formoterol fumarate and fluticasone propionate as medicaments, optionally including excipients, to form the combined doses.

3. The method according to claim 1, comprising the further step of
co-ordinating the combined doses such that when the combined doses are introduced for inhalation in the inhaler device adapted for prolonged delivery, the metered entities of a formoterol dose are sucked up first and the metered entities of a fluticasone dose are sucked up thereafter, whereby formoterol powder and fluticasone powder will be separately deposited in the lungs of a user.

4. The method according to claim 2, comprising the further step of
co-ordinating the combined doses such that when the combined doses are introduced for inhalation in the inhaler device adapted for prolonged delivery, the metered entities of a formoterol dose are sucked up first and the metered entities of a fluticasone dose are sucked up thereafter, whereby formoterol powder and fluticasone powder will be separately deposited in the lungs of a user.

5. The method according to claim 1 or 2, characterized by the further step of
co-ordinating the combined doses such that when the combined doses are introduced for inhalation in the adapted inhaler device, the metered entities of a formoterol dose are sucked up together with the metered entities of a fluticasone dose, whereby the medication powders during a prolonged delivery enter into the airways of a user as a mixed aerosol.

6. The method according to claim 1, comprising the further step of
co-ordinating the combined doses such that when the combined doses are introduced for inhalation in the adapted inhaler device, the metered entities of a formoterol dose are sucked up together with the metered entities of a fluticasone dose, whereby the medication powders during a prolonged delivery enter into the airways of a user as a mixed aerosol.

7. A method according to claim 2, comprising the further step of
preparing metered dry powder medicinal combined doses comprising separately deposited entities of the medicaments, where aerodynamic particle size for formoterol is generally in a range of 1 to 5 μm and for fluticasone generally in a range of 2 to 8 μm;
co-ordinating the combined doses such that the entities of the formoterol dose are sucked up first and the entities of the fluticasone dose are sucked up thereafter, whereby the formoterol dose will be deposited in a more peripheral portion of a lung and the fluticasone dose will be deposited in a more central portion of the lung.

8. The method according to claim 1, comprising the further step of
preparing the dry powder medicinal combined doses to provide a total mass in a range from 5 μg to 50 mg.

9. The method according to claim 2, comprising the further step of
preparing the dry powder medicinal combined doses to provide a total mass in a range from 5 μg to 50 mg.

10. The method according to claim 1, comprising the further step of
separating deposited entities of the included medicaments from each other onto a dose bed, intended for introduction into the adapted inhaler device, such that the medicaments cannot detrimentally mix with each other after forming of the combined doses.

11. The method according to claim 2, comprising the further step of
separating deposited entities of the included medicaments from each other onto a dose bed, intended for introduction into the adapted inhaler device, such that the medicaments cannot detrimentally mix with each other after forming of the combined doses.

12. Combined doses of pharmaceutical dry powders, adapted for administration by inhalation using a dry powder inhaler device (DPI), said inhaler device designed for a prolonged delivery of the combined doses, wherein
medicaments (A) and (B) are selected for a forming of pharmaceutical, combined doses, where (A) stands for formoterol or a pharmaceutically acceptable salt, enantiomer, racemate, hydrate, or solvate including mixtures thereof, and (B) stands for fluticasone or a pharmaceutically acceptable salt, enantiomer, racemate, hydrate, or solvate including mixtures thereof, and where (A) and (B) may optionally further include excipients;
the combined doses of pharmaceutical dry powders are prepared to comprise separate, deposited entities of medicinally effective quantities of the selected medicaments respectively onto selected target areas of a common dose bed, intended for introduction into the dry powder inhaler device, the sum of the deposited entities constituting metered quantities of powder in the combined doses of pharmaceutical dry powders;
the entities of the combined doses are co-ordinated during preparation such that, when the combined doses have been introduced into an inhaler device adapted for a prolonged delivery and when suction is applied through the inhaler device, the powders of each of the entities are gradually aerosolized, whereby the entities of the combined doses are delivered to and deposited in the lungs either simultaneously or separately in sequence, or in a combination thereof, during a single inhalation effort by a user.

13. The combined doses according to claim 12, wherein
formoterol fumarate and fluticasone propionate are selected as medicaments, optionally including excipients, to form the combined doses.

14. The combined doses according to claim 12, wherein
the combined doses are co-ordinated such that, when the combined doses are introduced for inhalation in said inhaler device adapted for a prolonged delivery, the metered entities of a formoterol dose are sucked up first and the metered entities of a fluticasone dose are sucked up thereafter, whereby formoterol powder and fluticasone powder are separately deposited in the lungs of a user.

15. The combined doses according to claim 13, wherein
the combined doses are co-ordinated such that, when the combined doses are introduced for inhalation in said inhaler device adapted for a prolonged delivery, the metered entities of a formoterol dose are sucked up first and the metered entities of a fluticasone dose are sucked up thereafter, whereby formoterol powder and fluticasone powder are separately deposited in the lungs of a user.

16. The combined doses according to claim 12, wherein
the combined doses are co-ordinated such that when the combined doses are introduced for inhalation through said inhaler device adapted for prolonged delivery, the metered entities of a formoterol dose are sucked up together with the metered entities of a fluticasone dose, whereupon the medication powders enter into the airways of a user as a mixed aerosol.

17. The combined doses according to claim 13, wherein
the combined doses are co-ordinated such that when the combined doses are introduced for inhalation through said inhaler device adapted for prolonged delivery, the metered entities of a formoterol dose are sucked up together with the metered entities of a fluticasone dose, whereupon the medication powders enter into the airways of a user as a mixed aerosol.

18. The combined doses according to claim 12, wherein
the combined doses are prepared to provide a total mass in a range from 5 μg to 50 mg.

19. The combined doses according to claim 13, wherein
deposited metered entities of medicaments are separated from each other onto a dose bed, such that the medicaments cannot detrimentally mix with each other after forming of the combined doses.

20. A use of different dry powder asthma medicaments having different therapeutic effects in a user, said dry powder asthma medicaments arranged for combined doses adapted for inhalation by a user of an inhaler device, said inhaler device being designed for a prolonged delivery of the combined doses, wherein
medicaments (A) and (B) are selected for a forming of pharmaceutical, combined doses, where (A) stands for formoterol or a pharmaceutically acceptable salt, enantiomer, racemate, hydrate, or solvate including mixtures thereof, and (B) stands for fluticasone or a pharmaceutically acceptable salt, enantiomer, racemate, hydrate, or solvate including mixtures thereof, and where (A) and (B) may optionally further include excipients;
a suitable pattern of physical positions and extensions in space is selected for the deposition onto a common dose bed of separate, metered powder entities of selected medicaments (A) and (B) respectively constituting the combined doses;
the entities of the combined doses are coordinated during preparation such that, when the combined doses have been introduced into an inhaler device adapted for a prolonged delivery and suction is applied through the inhaler device, the powders of each of the entities are gradually aerosolized, whereby the entities of the combined doses are delivered to and deposited in the lungs either simultaneously or separately in sequence, or in a combination thereof, during a single inhalation effort by a user, and
the delivered formoterol dose has a fine particle fraction (FPF) of at least 40%, preferably at least 50%, by mass and is generally deposited in the peripheral lung and the delivered fluticasone dose has a fine particle fraction (FPF) of at least 40%, preferably at least 50%, by mass and is generally deposited in the central lung, whereby optimized therapeutic effects and minimized adverse side effects can be attained.

21. The use according to claim 20, wherein
formoterol fumarate and fluticasone propionate are selected as medicament (A) and (B) respectively, optionally including excipients, in forming the combined doses.

22. The use according to claim 20, wherein
metered dry powder medicinal combined doses are prepared comprising separately deposited entities of said medicaments, where aerodynamic particle size for formoterol is generally in a range 1 to 5 μm and for fluticasone generally in a range 2 to 8 μm;

23. The use according to claim 20, wherein
said entities of formoterol and fluticasone are coordinated during preparation such that a formoterol dose will be sucked up first followed by a fluticasone dose thereafter, generally without mixing, when delivered to a user of the inhaler device;

24. The use according to claim 20, wherein
said combined doses are coordinated such that when the combined doses are introduced for inhalation through the inhaler device adapted for prolonged delivery, the metered entities of a formoterol dose are sucked up together with the metered entities of a fluticasone dose, whereupon the medication powders enter into the airways of a user as a mixed aerosol.

25. The use according to claim 20, wherein
the dry powder medicinal combined doses are prepared to a total mass in a range from 5 μg to 50 mg.

26. The use according to claim 20, wherein
entities of the medicament combined doses are separated during preparation from each other onto a common dose bed, such that the medicaments cannot detrimentally mix with each other after forming of the combined doses onto the dose bed.
